(19) United States
(12) Patent Application Publication
(10) Pub. No.: US 2009/0071600 A1
(43) Pub. Date: Mar. 19, 2009

(54) METHOD FOR PRODUCING DISPOSABLE WORN ARTICLE

(76) Inventor: Takao Wada, Osaka (JP)

Correspondence Address: MARK D. SARALINO (GENERAL);RENNER, OTTO, BOISSELLE & SKLAR, LLP, 1621 EUCLID AVENUE, NINETEENTH FLOOR, CLEVELAND, OH, 44115-2191, US

(21) Appl. No.: 12/298,783

(22) PCT Filed: Jun. 7, 2007
(86) PCT No.: PCT/JP2007/061544

§ 371 Date: Oct. 28, 2008

(30) Foreign Application Priority Data
Jun. 13, 2006 (JP) 2006-163318
Jul. 19, 2006 (JP) 2006-196443

(51) Int. Cl. B32B 38/04; A61F 13/15

(52) U.S. Cl. 156/259; 604/385.01; 604/392

(57) ABSTRACT

A method for producing a T-shaped worn article, which produces less wasted material. A web W is slit along a predetermined first cut-off line CL, thereby separating the web W into a first separate sheet W1 and a second separate sheet W2. The first separate sheet W1 and the second separate sheet W2 are spaced apart from each other so that the first separate sheet W1 and the second separate sheet W2 are apart from each other. A core portion 2 is placed so as to bridge between the first separate sheet W1 and the second separate sheet W2, thereby producing a composite material 10. The material is cut off along a second cut-off line Ch extending across the separate sheets W1 and W2 so that a portion thereof to be a back portion 3L is longer than a portion thereof to be a front portion 3S in the carrying direction X of the separate sheets W1 and W2.

### TECHNICAL FIELD

The present invention relates to a method for producing a worn article, such as a so-called “T-shaped” (belt-type) diaper.

### BACKGROUND ART

The first patent document, identified below, discloses a method for producing such a T-shaped diaper in a so-called “lateral flow process”.

The second patent document, identified below, discloses a method for producing an H-shaped diaper or pants in a lateral flow process.

[First Patent Document] Japanese National Phase PCT Laid-Open Publication No. 2005-519675 (Abstract)

[Second Patent Document] WO2005/004776A1 (FIG. 5)

### DISCLOSURE OF THE INVENTION

In the invention of the first patent document, a composite material is cut and divided into worn articles in such a manner that the back portion is longer than the front portion, thereby producing a belt-type worn article.

In the first patent document, however, the worn article is subjected to a die-cutting process to form leg holes, thus producing some wasted material.

The second patent document discloses the step of cutting a continuous web along a wave-shaped cut-off line, and the step of placing a core portion between two separate sheets obtained by the slitting.

However, the second patent document fails to disclose the process of cutting and dividing a composite material into individual products so that T-shaped diapers can be produced.

It is therefore an object of the present invention to provide a method for producing a T-shaped worn article, which produces less wasted material.

A first aspect of the present invention is directed to a method for producing a worn article, including a core portion covering a crotch of a wearer and a torso portion fitted around a torso of the wearer, wherein the torso portion includes a back portion and a front portion whose length in an torso direction is smaller than that of the back portion, and the core portion is placed so as to bridge between the back portion and the front portion, the method including the steps of slitting a strip-shaped sheet material being continuous in a carrying direction and having a first side edge portion and a second side edge portion along a predetermined first cut-off line, thereby separating the strip-shaped sheet material into a first separate sheet and a second separate sheet; spacing apart the first separate sheet and the second separate sheet from each other so that the first cut-off line on the first separate sheet and the first cut-off line on the second separate sheet are apart from each other; placing the core portion so as to bridge between the first separate sheet and the second separate sheet, thereby forming a composite material; and cutting off the separate sheets along a second cut-off line extending across the separate sheets so that a portion thereof to be the back portion is longer than a portion thereof to be the front portion in a carrying direction of the separate sheets, so that the front portion and the back portion are formed alternately from the first separate sheet, and so that the back portion and the front portion are formed alternately from the second separate sheet, thereby dividing the composite material into individual products.

According to the first aspect of the present invention, the core portion is placed between the first separate sheet and the second separate sheet, which are spaced apart from each other, whereby leg holes are formed by the spaces between the separate sheets. As a result, there is less wasted material.

Moreover, it is possible to produce a T-shaped diaper since the separate sheets are cut off along the second cut-off line so that a portion to be the back portion is longer than a portion to be the front portion in the carrying direction.

A second aspect of the present invention is directed to a method for producing a worn article, including a core portion covering a crotch of a wearer and a torso portion fitted around a torso of the wearer, wherein the torso portion includes a back portion and a front portion whose length in an torso direction is smaller than that of the back portion, and the core portion is placed so as to bridge between the back portion and the front portion, the method including the steps of slitting a strip-shaped sheet material being continuous in a carrying direction and having a first side edge portion and a second side edge portion along a predetermined wave-shaped first cut-off line, thereby separating the strip-shaped sheet material into a first separate sheet and a second separate sheet, the first separate sheet having first depressed portions and protruding portions alternating with each other, and the second separate sheet having second depressed portions and protruding portions alternating with each other; spacing apart the first separate sheet and the second separate sheet from each other so that the first cut-off line on the first separate sheet and the first cut-off line on the second separate sheet are apart from each other; placing a first core portion so as to bridge between one of the first protruding portions of the first separate sheet and one of the second depressed portions of the second separate sheet, and placing a second core portion so as to bridge between one of the second protruding portions of the second separate sheet and one of the first depressed portions of the first separate sheet, thereby forming a composite material; and cutting off the separate sheets along a second cut-off line extending across the separate sheets so that the front portion is formed from the first or second depressed portion and the back portion including the first or second protruding portion is formed, so that a portion thereof to be the back portion is longer than a portion thereof to be the front portion in a carrying direction of the separate sheets, so that the front portion and the back portion are formed alternately from the first separate sheet, and so that the back portion and the front portion are formed alternately from the second separate sheet, thereby dividing the composite material into individual products.

According to the second aspect of the present invention, there is provided an effect as follows in addition to those provided by the first aspect of the present invention.

That is, with the web being cut off along a wave-shaped first cut-off line, a part of the back portion is produced from the protruding portion, and the front portion is produced from the depressed portion.

As a result, it is possible to produce, with no wasted material, a T-shaped worn article of which the back portion can sufficiently cover the buttocks and the front portion is small.

In a preferred embodiment of the present invention, the first cut-off line is determined so that a width of the portion to be the back portion in a width direction perpendicular to the carrying direction of the back portion gradually decreases toward the portion to be the front portion adjacent to the back portion, and the web is slit along the first cut-off line.

In this embodiment, it is possible to produce a T-shaped worn article having such a shape that the hips are sufficiently covered.

Moreover, the back portion to be attached to the front portion is narrow, providing desirable wearability.

In a preferred embodiment of the present invention, the method further includes the step of trimming a part of the sheet material so as to reduce an area to cover buttocks of the wearer.

In this embodiment, the area to cover the buttocks will not be larger than necessary.

In another preferred embodiment of the present invention, the method further includes the step of attaching flaps, each to be a part of the torso portion, to opposite ends in the carrying direction of a part to be the back portion of the sheet material, the separate sheets or the composite material.

In this embodiment, the wing portions of the back portion become longer by the lengths of the flaps attached thereto.

Therefore, it is possible to keep the amount of trimming small even with adult diapers having large torsos.

### BRIEF DESCRIPTION OF THE DRAWINGS

FIG. 1 is a plan view showing an example of a T-shaped diaper produced by a production method according to Embodiment 1 of the present invention.

FIG. 2A is a cross-sectional view taken along line IIA-IIA of FIG. 1, and FIG. 2B is a cross-sectional view taken along line IIB-IIB of FIG. 1.

FIG. 3A is a front view showing a diaper being worn, and

FIG. 3B is a front view showing a worn diaper.

FIG. 4A and FIG. 4B are schematic plan views each showing a production method.

FIG. 5A is a schematic plan view showing a production method, FIG. 5B, FIG. 5C and FIG. 5D are plan views each showing how a diaper is folded.

FIG. 6 is a plan view showing a part of a production method of Embodiment 2, and an example of a T-shaped diaper produced by this method.

FIG. 7 is a plan view showing an example of a T-shaped diaper produced by a production method of Embodiment 3.

FIG. 8A and FIG. 8B are cross-sectional views each showing a diaper while not showing cuffs, wherein FIG. 8A is a cross-sectional view taken along line VIIIA-VIIIA of FIG. 7, and

FIG. 8B is a cross-sectional view taken along line VIIIB-VIIIB of FIG. 7.

FIG. 9 is a plan view showing a composite material being produced.

FIG. 10A and FIG. 10B are plan views each showing a variation of a flap.

### DESCRIPTION OF THE REFERENCE NUMERALS

1: Diaper
2: Core portion
3: Torso portion
3L: Back portion
3S: Front portion
10: Composite material
CL: First cut-off line
Ch: Second cut-off line
W: Sheet material
W1s: First side edge portion
W2s: Second side edge portion
W1: First separate sheet
W2: Second separate sheet
W1d: First depressed portion
W1u: First protruding portion
W2d: Second depressed portion
W2u: Second protruding portion
X: Carrying direction
D: Width direction

### BEST MODE FOR CARRYING OUT THE INVENTION

The present invention will be understood more clearly from the following description of preferred embodiments taken in conjunction with the accompanying drawings. Note however that the embodiments and the drawings are merely illustrative and should not be taken to define the scope of the present invention. The scope of the present invention shall be defined only by the appended claims. In the accompanying drawings, like reference numerals denote like components throughout the plurality of figures.

One embodiment of the present invention will now be described with reference to the drawings.

First, an example of a diaper that can be produced by the present production method will now be described before describing the production method of the present embodiment.

### EMBODIMENT 1

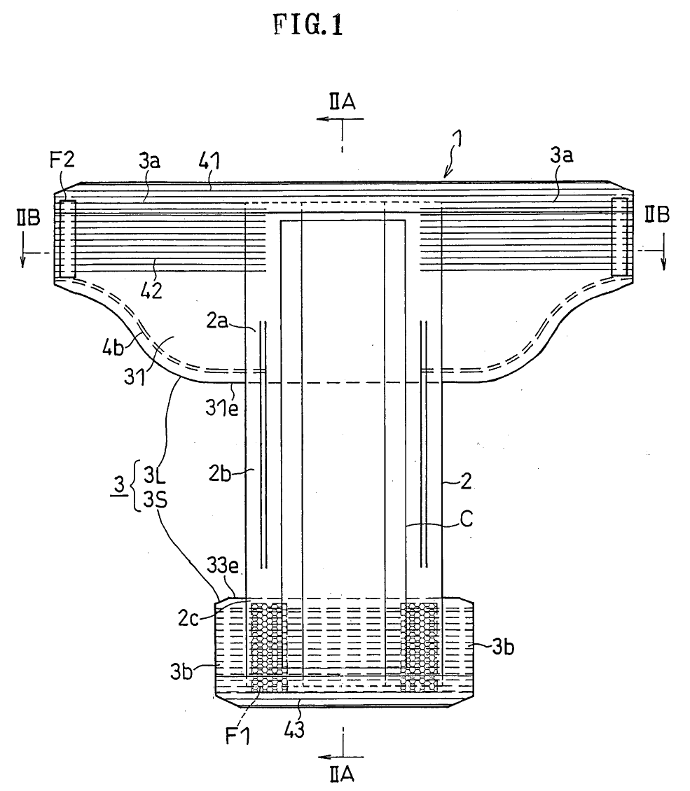

Referring to FIG. 1, a diaper 1 includes, as an integral part, a core portion 2 and a torso portion 3 whose length is greater than the width of the core portion 2.

The torso portion 3 includes a back portion 3L that is longer in the torso direction, and a front portion 3S that is shorter in the torso direction than the back portion 3L.

The core portion 2 is placed so as to bridge between the back portion 3L and the front portion 3S.

The shape of a lower edge 31e of a hip portion 31 of the back portion 3L generally conforms to that of a lower edge 33e of the front portion 3S.

In the diaper 1, the core portion 2 includes, as an integral part, a base portion 2a laid on and attached to the back portion 3L, a crotch portion 2b to be placed over the crotch of the wearer, and a tip portion 2c laid on and attached to the front portion 3S.

In other words, a part of the core portion 2 between the base portion 2a laid on the back portion 3L and the tip portion 2c laid on the front portion 3S forms the crotch portion 2b, which covers the crotch.

The base portion 2a and the tip portion 2c of the core portion 2 may form a part of the torso portion.

Wing portions 3a and 3a protruding beyond the core portion 2 in the left-right direction are integrally provided on opposite end portions of the back portion 3L.

When wearing the diaper 1, the back portion 3L is put around the torso of the wearer, and the wing portions 3a and 3a are attached to the front portion 3S.

Tab portions 3b and 3b protruding beyond the core portion 2 in the left-right direction are provided on opposite end portions of the front portion 3S.

When wearing the diaper 1, the wing portions 3a and 3a can be attached by picking the tab portion 3b between fingers and pulling the back portion 3L.

First touch fasteners F1 are attached to the front portion 3S.

Second touch fasteners F2, which can be touch-fastened with the first touch fasteners F1, are attached to the opposite wing portions 3a and 3a of the back portion 3L.

The first touch fasteners F1 may be omitted. In such a case, the outermost sheet of the core portion 2 or the front portion 3S serves as the female touch fastener.

The touch fasteners F1 and F2 may be replaced by an adhesive tape and a portion onto which the adhesive tape is applied.

The core portion 2 includes an absorbent body C capable of absorbing body fluids discharged from within the body.

The core portion 2 may be so long that it reaches a part of the area of the back portion 3L or the front portion 3S.

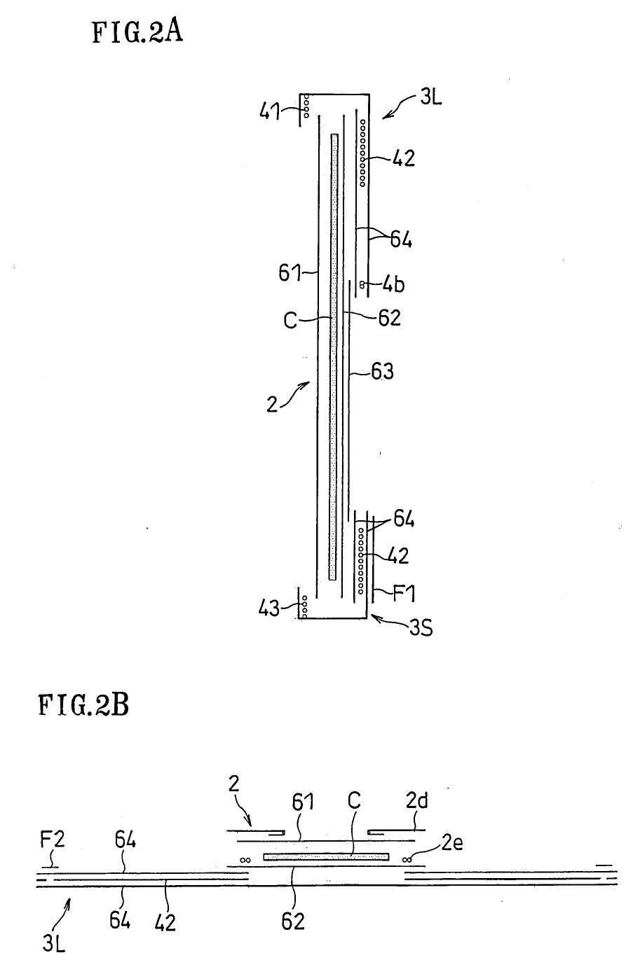

As shown in FIG. 2A, in the back portion 3L or the front portion 3S, the end portion of the core portion 2 may be held down by a sheet.

The absorbent body C may include a fluff pulp obtained by milling a pulp and/or a super absorbing polymer. The absorbent body C may be an air-laid pulp, or the like.

As shown in FIG. 2B, the absorbent body C may be provided with a plurality of anti-leak walls (so-called “cuffs”) 2d for preventing the feces and/or urine from leaking to the outside of the absorbent body C or the diaper. The anti-leak wall 2d may have a three-dimensional configuration.

The core portion 2 may be provided with a plurality of leg gathers 2e for preventing the feces and/or urine from leaking out of the diaper.

Moreover, an elastic member 4b may be provided for forming other leg gathers curved along the lower edge 31e of the hip portion 31 of the back portion 3L of FIG. 1.

Elastic members 41 and 43 are placed on the back portion 3L and the front portion 3S so as to form waist gathers for suppressing the slippage between the wearer and the diaper 1.

Moreover, elastic members 42 to be fit gathers are placed on the back portion 3L in order to improve the fit of the diaper 1.

The elastic members 41 to 43 and 4b may be rubber threads or rubber tapes. The elastic members 41 to 43 and 4b may be made of urethane, and a web having elasticity may be used for the back portion 3L and/or the front portion 3S.

In order to prevent the corrugation of the touch fasteners and the absorbent body to be described later, the elastic members 41 to 43 and 4b may be partially omitted or may be cut off, for example, so that the elasticity is partially lost.

As shown in FIGS. 1, 2A and 2B, the core portion 2 includes a liquid-permeable top sheet 61, the absorbent body C, a liquid-impermeable back sheet 62, and an outer sheet 63 layered together. The anti-leak walls 2d are provided on the top sheet 61.

As shown in FIGS. 1, 2A and 2B, the back portion 3L and the front portion 3S include a cover sheet 64 made of non-woven fabric and the elastic member 42 layered together.

The cover sheet 64 is folded at the waist portion of the back portion 3L and the front portion 3S. The cover sheet 64 has two layers with the elastic member 42 being interposed therebetween.

Production Method:

An example of a method for producing a worn article will now be described.

Step of Producing Composite Sheet Ws:

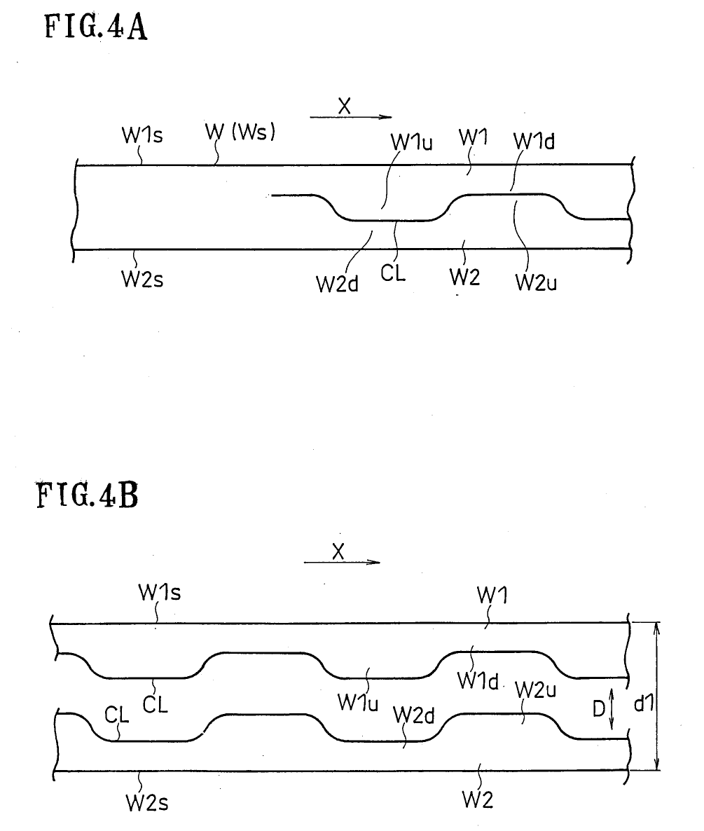

As shown in FIG. 4A, a strip-shaped web W being continuous in the carrying direction X has first and second side edge portions W1s and W2s and is carried in the carrying direction X.

An elastic member (not shown) is placed in a straight line pattern or a wave-shaped pattern on the web W being carried, thereby forming a composite sheet Ws.

The composite sheet Ws may be formed by using a pair of webs W and W with an elastic member being interposed therebetween. The elastic member (not shown) may be placed along a first cut-off line CL, along which the composite sheet Ws is later slit.

Division Step:

The composite sheet Ws being carried is cut off along the first cut-off line CL continuous in the carrying direction X of the composite sheet Ws.

As the composite sheet Ws is cut off, the composite sheet Ws is divided into first and second separate sheets W1 and W2.

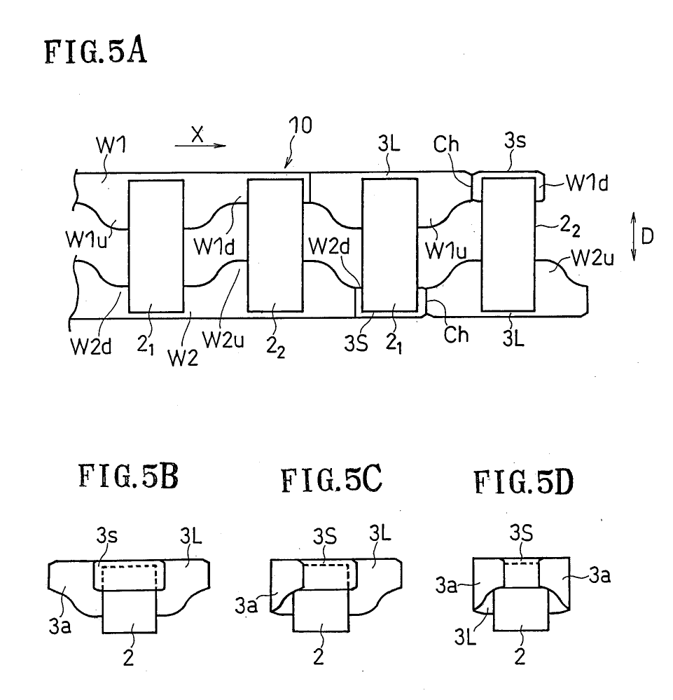

In the portion to be the back portion 3L in FIG. 1, the composite sheet Ws of FIG. 4A is slit along the first cut-off line CL so that the width of the back portion 3L in the width direction D perpendicular to the carrying direction X gradually decreases toward the portion to be the front portion 3S adjacent to the back portion 3L in FIG. 5A.

As the sheet is slit as described above, it is possible to alternately form the front portions 3S and the back portions 3L of FIG. 5A in the two separate sheets W1 and W2, with no wasted material.

The first separate sheet W1 of FIG. 4A has the first side edge portion W1s, and includes first protruding portions W1u and first depressed portions W1d alternating with each other along the first cut-off line CL.

The second separate sheet W2 has the second side edge portion W2s, and includes second depressed portions W2d and second protruding portions W2u alternating with each other along the first cut-off line CL.

Separation Step:

After the division step, the two separate sheets W1 and W2 are spaced apart from each other in the width direction D so that the width dl from the first side edge portion W1s to the second side edge portion W2s increases, as shown in FIG. 4B.

In other words, the first separate sheet W1 and the second separate sheet W2 are spaced apart from each other so that the first cut-off line CL on the first separate sheet W1 and the first cut-off line CL on the second separate sheet W2 are apart from each other.

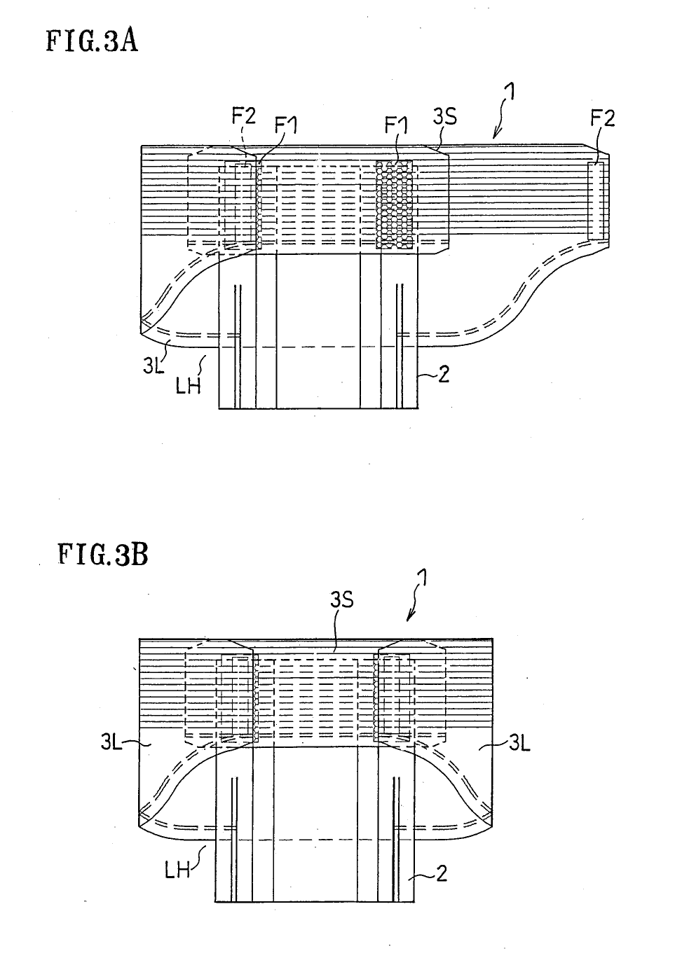

A part of the space between the first separate sheet W1 and the second separate sheet W2 created by the division step forms a part or whole of a leg hole LH (FIG. 3B) in the product.

Production of Composite Material:

Then, as shown in FIG. 5A, the core portions 2 are placed on the separate sheets W1 and W2, thereby producing a composite material 10.

Specifically, a first core portion 2₁ is placed so as to bridge between the first protruding portion W1u of the first separate sheet W1 and the second depressed portion W2d of the second separate sheet W2.

A second core portion 2₂ is placed so as to bridge between the second protruding portion W2u of the second separate sheet W2 and the first depressed portion W1d of the first separate sheet W1.

Immediately after, or at the same time as, the placement step, the core portions 2₁ and 2₂ are attached to the protruding portions W1u and W2u and the depressed portions W1d and W2d.

Cutting and Dividing:

After the attachment step, the separate sheets W1 and W2 are cut off along a second cut-off line Ch extending across the separate sheets W1 and W2. As a result, the composite material 10 is cut and divided into individual diapers 1.

In this cutting and dividing step, the first separate sheet W1 is cut off at a position in the vicinity of the second core portion 2₂. The second separate sheet W2 is cut off at a position in the vicinity of the first core portion 2₁.

Thus, in this cutting and dividing step, the front portion 3S is formed from the first or second depressed portion W1d, W2d, and the back portion 3L is formed including the first or second protruding portion W1u, W2u.

Moreover, the separate sheets W1 and W2 are cut off so that a portion thereof to be the back portion 3L is longer than a portion thereof to be the front portion 3S in the carrying direction X of the separate sheets W1 and W2.

Moreover, the separate sheets W1 and W2 are cut off so that the front portions 3S and the back portions 3L are formed alternately from the first separate sheet W1 and so that the back portions 3L and the front portions 3S are formed alternately from the second separate sheet W2.

In the cutting and dividing step, the first separate sheet W1, the second separate sheet W2 or the core portions 2₁ and 2₂ may be partially trimmed.

Folding:

The cut and divided diapers 1 are held and carried downstream by one or more holding means such as suction pads (not shown), and are folded as follows on the holding means while being carried.

Specifically, first, the core portion 2 is folded in two so that the front portion 3S is laid on the back portion 3L, as shown in FIG. 5B.

Then, as shown in FIG. 5C, a portion of one wing portion 3a is folded so as to be laid on a portion of the front portion 3S.

Moreover, as shown in FIG. 5D, a portion of the other wing portion 3a is folded so as to be laid on a portion of the front portion 3S.

The method for folding the back portion 3L may be any method known in the art such as, for example, those disclosed in Japanese Laid-Open Patent Publication No. 2005-000296 and 2004-248785.

In the folding step, the second touch fastener F2 may be engaged with the first touch fastener F1, as shown in FIGS. 3A and 3B.

Although the sheet material W is slit along a wave-shaped first cut-off line in the embodiment above, the sheet material W may be slit along a straight first cut-off line.

Also in such a case, unnecessary portions may be trimmed off after the slitting step. As compared with the technique of the first patent document in which leg holes are formed in a die-cutting process, such trimming is unlikely to produce wasted material.

### EMBODIMENT 2

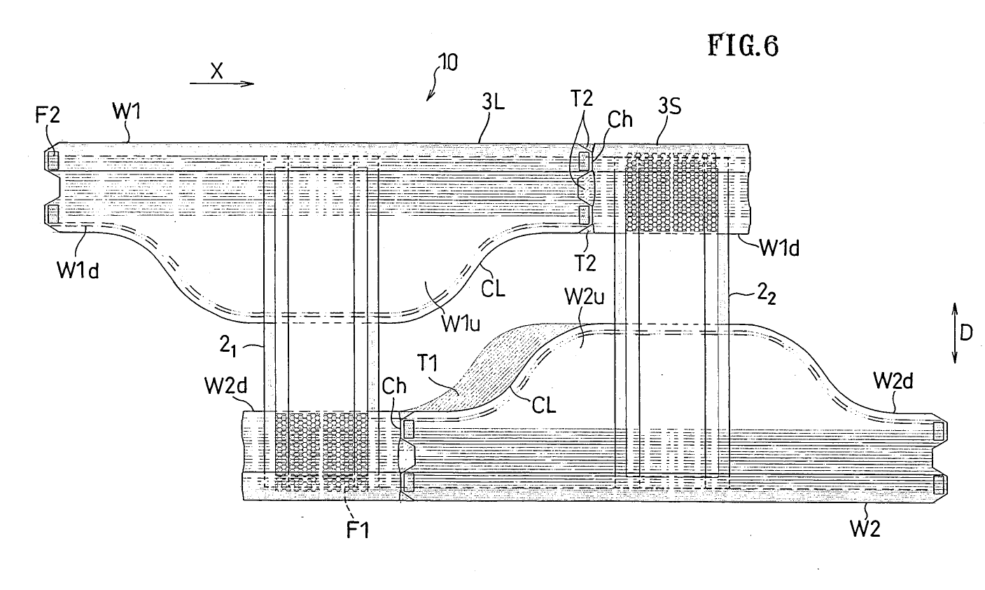

FIG. 6 shows Embodiment 2.

This embodiment further includes, in addition to the steps of the method of Embodiment 1, a trimming step of trimming a portion T1 of the sheet material so as to reduce the area to cover the buttocks of the wearer.

The first trimmed portion T1 is formed by cutting off the portion between the first cut-off line CL on the first separate sheet W1 and the first cut-off line CL on the second separate sheet W2, which are opposing each other.

The trimming step of clipping the first trimmed portion T1 may be performed at any timing.

However, the trimming step is preferably performed at the same time as the step of dividing the sheet material W. This is because a single cutter drum can be then shared among the cutting operations.

In the present production method, a second trimmed portion T2 may be trimmed.

The second trimmed portion T2 is formed by cutting off portions between the front portion 3S of a diaper and the back portion 3L of an adjacent diaper.

With this trimming step, the front portion 3S and the back portion 3L will have desirably-shaped edges.

### EMBODIMENT 3

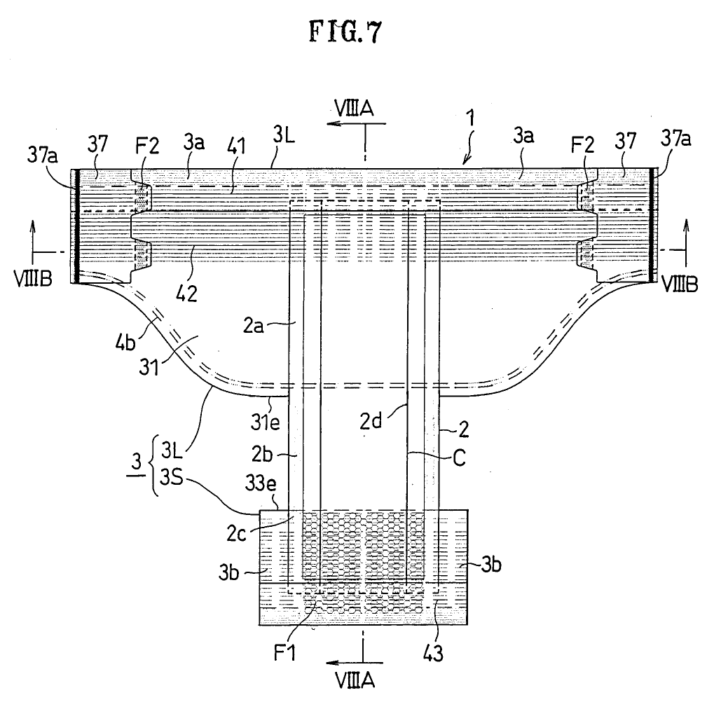

FIGS. 7 to 9 show Embodiment 3. The structure of the diaper of Embodiment 3 will be described.

In the present embodiment, flaps 37 are attached to opposite end portions of the wing portions 3a and 3a in the torso direction.

The flaps 37 increase the effective length of the back portion 3L in the torso direction.

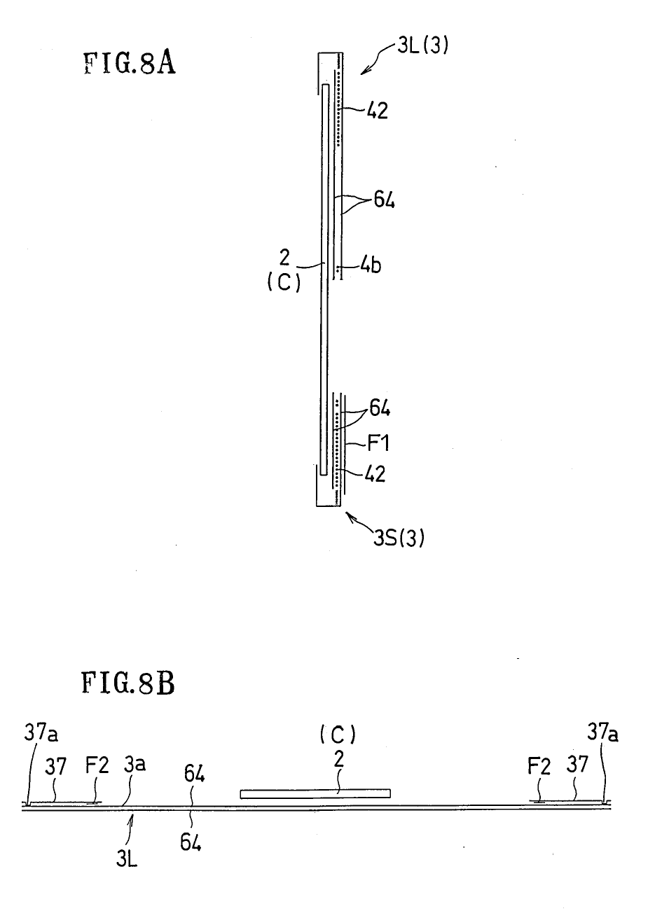

As is clearly shown in FIG. 8B, the flap 37 is attached to the wing portion 3a along an attachment line 37a at the proximal end of the wing portion 3a in the torso direction.

The flap 37 extends in the torso direction from the attachment line 37a toward the core portion 2.

The second touch fastener F2 is attached to the end of the flap 37 that is opposite to the attachment line 37a. The second touch fastener F2 is engaged with the inner surface of the back portion 3L, whereby the wing portion 3a and the flap 37 are tentatively attached together.

When wearing the diaper, the tentative attachment between the second touch fastener F2 and the wing portion 3a is disengaged, and the back portion 3L is put around the torso, after which the second touch fastener F2 is engaged with the first touch fastener F1 of FIG. 8A, thus fitting the torso portion 3 around the torso.

In FIGS. 8A and 8B, the cuffs 2d are not shown.

Next, the diaper production method of Embodiment 3 will be described.

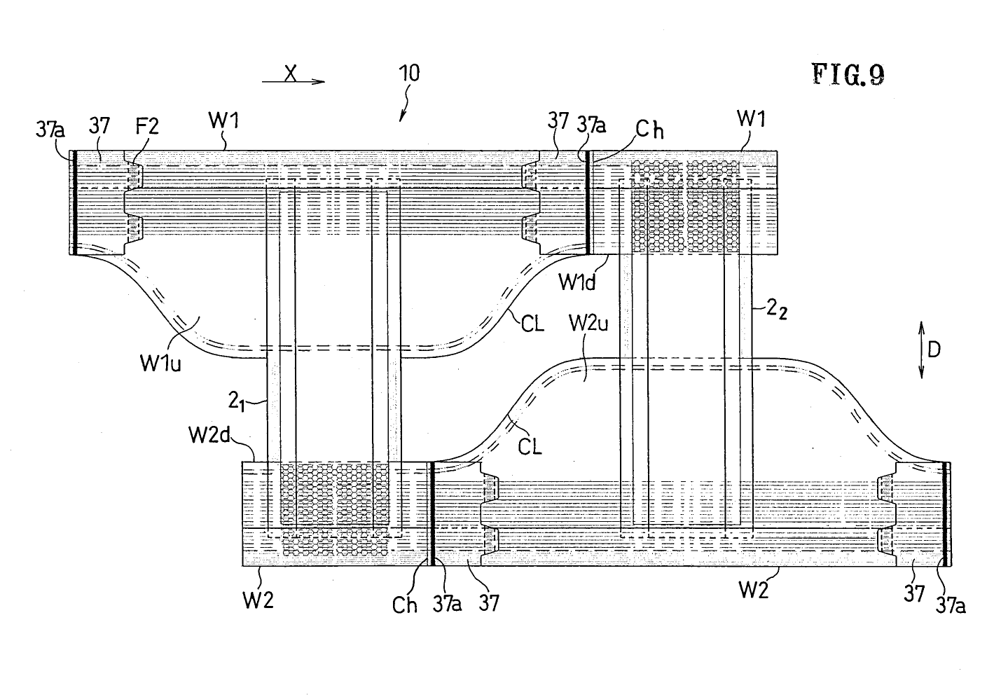

The method of the present embodiment further includes, in addition to the steps of the method of Embodiment 1, the fixing step of fixing (attaching) the flap 37 shown in FIG. 9 to the wing portion 3a.

The fixing step may be performed at any timing, before the division of the sheet material W shown in FIG. 4A, before the formation of the composite material 10 shown in FIG. 5A after the division step, or after the formation of the composite material 10 shown in FIG. 9. In other words, it is only required that the flaps 37 are attached to the opposite ends, in the carrying direction X, of a portion of the sheet material W (FIG. 4A), each separate sheets W1 and W2 or the composite material 10 that is to be the back portion 3L.

The method for fixing the flap 37 may be any of various methods such as welding and bonding.

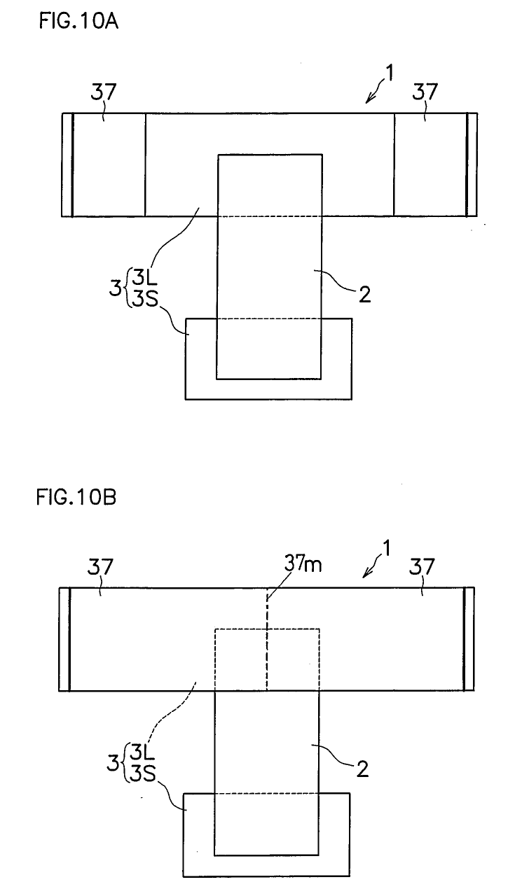

The shape of the flap 37 may be any of various shapes, e.g., a square shape shown in FIG. 10A, or a strip shape with tearable perforations 37m shown in FIG. 10B.

The flaps 37 may cover a part of the core portion 2.

Particularly in a case where the first cut-off line CL is an S-shaped (wave-shaped) line, the curve becomes milder if the pitch between the adjacent core portions 2 is widened as shown in FIG. 6.

Then, the area to cover the buttocks will become larger than necessary. As a result, it is necessary to trim the extra portion T1 as shown in FIG. 6.

In contrast, in the embodiment of FIG. 9, the curve is sharper as shown by the first cut-off line CL of FIG. 9, as compared with the first cut-off line CL of FIG. 6. Therefore, the area to cover the buttocks will not be larger than necessary.

As a result, it is possible to eliminate the need for the trimming, or to reduce the first trimmed portion T1 (FIG. 6).

Such a technical concept of Embodiment 3 is applicable not only to T-shaped diapers but also to H-shaped diapers.

In the embodiments shown in FIGS. 6 to 9, the configurations and the steps other than those discussed above are as described above in the method of Embodiment 1, and like elements to those of Embodiment 1 will be denoted by like reference numerals and will not be further described below.

While preferred embodiments have been described above with reference to the drawings, various obvious changes and modifications will readily occur to those skilled in the art upon reading the present specification.

For example, pants may be produced, instead of diapers, by fixing the front portion and the back portion to each other by applying a hot-melt, an ultrasonic seal, or the like, between their end portions in the torso direction.

Elastic members in the front portion, or the like, may be omitted, and no torso elastic member may be provided.

The leg gathers may not be provided along the first cut-off line.

The core portion of the diaper may be folded in two after bending a belt.

Such changes and modifications shall be deemed to fall within the scope of the present invention, which is defined by the appended claims.

### INDUSTRIAL APPLICABILITY

The present invention is applicable to the production of disposable worn articles such as so-called “T-shaped diapers” or pants.

1. A method for producing a worn article, including a core portion covering a crotch of a wearer and a torso portion fitted around a torso of the wearer, wherein the torso portion includes a back portion and a front portion whose length in an torso direction is smaller than that of the back portion, and the core portion is placed so as to bridge between the back portion and the front portion, the method comprising the steps of:
slitting a strip-shaped sheet material being continuous in a carrying direction and having a first side edge portion and a second side edge portion along a predetermined first cut-off line, thereby separating the strip-shaped sheet material into a first separate sheet and a second separate sheet;
spacing apart the first separate sheet and the second separate sheet from each other so that the first cut-off line on the first separate sheet and the first cut-off line on the second separate sheet are apart from each other;
placing the core portion so as to bridge between the first separate sheet and the second separate sheet, thereby forming a composite material; and
cutting off the separate sheets along a second cut-off line extending across the separate sheets so that a portion thereof to be the back portion is longer than a portion thereof to be the front portion in a carrying direction of the separate sheets, so that the front portion and the back portion are formed alternately from the first separate sheet, and so that the back portion and the front portion are formed alternately from the second separate sheet, thereby dividing the composite material into individual products.

2. A method for producing a worn article, including a core portion covering a crotch of a wearer and a torso portion fitted around a torso of the wearer, wherein the torso portion includes a back portion and a front portion whose length in an torso direction is smaller than that of the back portion, and the core portion is placed so as to bridge between the back portion and the front portion, the method comprising the steps of:
slitting a strip-shaped sheet material being continuous in a carrying direction and having a first side edge portion and a second side edge portion along a predetermined wave-shaped first cut-off line, thereby separating the strip-shaped sheet material into a first separate sheet and a second separate sheet, the first separate sheet having first depressed portions and first protruding portions alternating with each other, and the second separate sheet having second depressed portions and second protruding portions alternating with each other;
spacing apart the first separate sheet and the second separate sheet from each other so that the first cut-off line on the first separate sheet and the first cut-off line on the second separate sheet are apart from each other;
placing a first core portion so as to bridge between one of the first protruding portions of the first separate sheet and one of the second depressed portions of the second separate sheet, and placing a second core portion so as to bridge between one of the second protruding portions of the second separate sheet and one of the first depressed portions of the first separate sheet, thereby forming a composite material; and
cutting off the separate sheets along a second cut-off line extending across the separate sheets so that the front portion is formed from each depressed portion and the back portion including the first or second protruding portion is formed, so that a portion thereof to be the back portion is longer than a portion thereof to be the front portion in a carrying direction of the separate sheets, so that the front portion and the back portion are formed alternately from the first separate sheet, and so that the back portion and the front portion are formed alternately from the second separate sheet, thereby dividing the composite material into individual products.

3. A method for producing a worn article according to claim 2, wherein the first cut-off line is determined so that a width of the portion to be the back portion in a width direction perpendicular to the carrying direction of the back portion gradually decreases toward the portion to be the front portion adjacent to the back portion, and the web is slit along the first cut-off line.

4. A method for producing a worn article according to claim 2, further comprising the step of trimming a part of the sheet material so as to reduce an area to cover buttocks of the wearer.

5. A method for producing a worn article according to claim 2, further comprising the step of attaching flaps, each to be a part of the torso portion, to opposite ends in the carrying direction of a part to be the back portion of the sheet material, the separate sheets or the composite material.
